(19) United States
(12) Patent Application Publication
(10) Pub. No.: US 2024/0003876 A1
(43) Pub. Date: Jan. 4, 2024

(54) LATERAL FLOW ASSAY CASSETTE WITH VERTICAL SWAB HOLDER

(71) Applicant: InBios International, Inc., Seattle, WA (US)

(72) Inventor: James William Needham, Seattle, WA (US)

(21) Appl. No.: 18/346,733

(22) Filed: Jul. 3, 2023

Related U.S. Application Data
(60) Provisional application No. 63/358,014, filed on Jul. 1, 2022.

(51) Int. Cl. G01N 33/543; G01N 33/569; G01N 33/68; B01L 3/00

(52) U.S. Cl. CPC G01N 33/54388; G01N 33/56911; G01N 33/6803; B01L 3/5029; G01N 33/56983; G01N 2800/26; B01L 2300/0609

(57) ABSTRACT

A lateral flow assay cassette including a vertical swab holder is provided herein. Also provided are kits including the lateral flow assay cassette and methods of detecting an analyte using the lateral flow assay cassette.

### PRIORITY CLAIM

The present application claims priority to U.S. Provisional Patent Application Ser. No. 63/358,014, filed Jul. 1, 2022, and titled “LATERAL FLOW ASSAY CASSETTE WITH VERTICAL SWAB HOLDER,” which is incorporated by reference herein in its entirety.

### BACKGROUND

Most assays that require a swab specimen (e.g., nasal, nasopharyngeal, oral) separately process the swab in order to extract the target (RNA, DNA, protein, etc.) prior to running the assay. This may include (but is not limited to) lysis steps, nucleic acid extraction steps and/or enrichment steps. For instance, current PCR testing for SCoV-2 typically requires swab specimens that are first placed in 1 to 3 mL of virus transport media (VTM). After transport to a suitable laboratory, RNA is subsequently extracted from the (now diluted) specimen. Another example of an immunoassay is the CL Detect™ rapid test produced by InBios International, Inc. in which, a ‘dental broach’ (i.e., a swab with barbs) is used to collect specimen from a lesion in the subject. The broach is incubated with lysis buffer in a separate tube for a period before a volume of the lysis buffer is then transferred onto the rapid test.

Still other immunoassays use swab specimens directly by placing them in the lateral plane of the assay in contact with a sample pad, then dropping an assay buffer over the swab.

### BRIEF SUMMARY

The disclosure provides the following embodiments.

Embodiment 1. A lateral flow assay cassette comprising a sample pad; a sample port that allows access to the sample pad; and a vertical swab holder that secures a swab having a stem and a head in a vertical position with respect to the cassette by engaging the head of the swab.

Embodiment 2. The lateral flow assay cassette of Embodiment 1, wherein the vertical swab holder includes an outer portion and an inner portion which contains the sample port in an orifice of the inner portion.

Embodiment 3. The lateral flow assay cassette of Embodiment 2, wherein the outer portion has a height that is greater than a height of the inner portion.

Embodiment 4. The lateral flow assay cassette of any one of Embodiments 1-3, wherein the vertical swab holder comprises a plurality of flexible elements that secure the swab in a vertical position.

Embodiment 5. The lateral flow assay cassette of Embodiment 4, wherein the plurality of flexible elements comprise a plurality of flexible protrusions that protrude from the inner portion.

Embodiment 6. The lateral flow assay cassette of Embodiment 4, wherein the plurality of flexible elements comprise a plurality of flexible ribs that protrude from the inner portion.

Embodiment 7. The lateral flow assay cassette of any one of Embodiments 1-3, wherein the vertical swab holder comprises a plurality of rigid elements that secure the swab in a vertical position.

Embodiment 8. The lateral flow assay cassette of Embodiment 7, wherein the plurality of rigid elements protrude from the inner portion.

Embodiment 9. The lateral flow assay cassette of any one of Embodiments 4-8, wherein the plurality of flexible elements or the plurality of rigid elements are sized to engage swab heads of varying dimensions.

Embodiment 10. A method of detecting an analyte, the method comprising: a) providing a lateral flow assay cassette having a sample pad, a sample port that allows access to the sample pad, and a vertical swab holder that secures a swab having a stem and a head in a vertical position with respect to the cassette by engaging the head of the swab; b) collecting a sample from a subject using the swab; c) placing the swab stem into the swab holder in a vertical position with respect to the cassette such that the swab head is in the sample port directly above the sample pad or directly in contact with the sample pad; and d) adding an appropriate amount of sample buffer to the swab head or vertical swab holder, or both.

Embodiment 11. The method of Embodiment 10, wherein the sample is from a nose, nasopharyngeal cavity, the oropharyngeal cavity, the mid-turbinate region, mouth, tongue, throat, teeth, gums, tonsils, outer ear canal, skin, wounds, lesions, urethra, vagina, cervix, anus, or rectum sample.

Embodiment 12. The method of Embodiment 10 or Embodiment 11, wherein the sample is a nasal or ear secretion, sputum, saliva, a vaginal secretion, pus, blood, urine, feces, a bronchoalveolar lavage sample, or a surgical sample.

Embodiment 13. The method of any one of Embodiments 10-12, wherein the detection of the analyte indicates the presence of an infectious agent in the subject.

Embodiment 14. The method of Embodiment 13, wherein the infectious agent is Human coronavirus, 229E; Human coronavirus, OC43; Human coronavirus, NL63; MERS-coronavirus; SARS-CoV-2; Adenovirus 21; Human Metapneumovirus (hMPV); Parainfluenza virus 1; Parainfluenza virus 2; Parainfluenza virus 3; Parainfluenza virus 4a; Influenza A; Influenza B; Enterovirus D68; Respiratory syncytial virus; Rhinovirus 40; Haemophilus influenza; Streptococcus pneumonia; Streptococcus pyogenes; Candida albicans; Bordetella pertussis; Mycoplasma pneumonia; Chlamydia pneumonia; Legionella pneumophila; or Staphylococcus aureus.

Embodiment 15. The method of any one of Embodiments 10-14, wherein the analyte is RNA, DNA, or protein.

Embodiment 16. The method of any one of Embodiments 10-15, further comprising detecting the analyte within 30 minutes of adding the sample buffer to the swab head or vertical swab holder, or both.

Embodiment 17. The method of any one of Embodiments 10-16, further comprising: e) introducing sample from the swab head to the sample pad; f) running the assay; and g) producing an assay result.

Embodiment 18. The method of any one of Embodiments 10-17, further comprising adding an appropriate amount of chase buffer to the swab head after step (d).

Embodiment 19. A kit comprising: a lateral flow assay cassette comprising: a sample pad; a sample port that allows access to the sample pad; and a vertical swab holder that secures a swab having a stem and a head in a vertical position with respect to the cassette by engaging the head of the swab; a swab having a stem with a diameter sized to be secured by the swab holder; and an instruction sheet.

Embodiment 20. The kit of Embodiment 19, further comprising sample buffer.

### BRIEF DESCRIPTION OF THE DRAWINGS

The disclosure may be better understood through reference to the figures, which illustrate example embodiments of the disclosure, and in which:

FIG. 1A and FIG. 1B are schematic top view diagrams of exemplary cassettes including vertical swab holders. FIG. 1A shows an exemplary cassette with a vertical swab holder having a plurality of flexible protrusions. FIG. 1B shows an exemplary cassette with a vertical swab holder having a plurality of ribs.

FIG. 2A is a schematic diagram of placement of a swab in the vertical swab holder of a cassette.

FIG. 2B is a schematic diagram of sample buffer being added after placement of a swab in the vertical swab holder.

FIG. 3A is a schematic diagram of a top view of three positive test results using the exemplary cassette of FIG. 1A.

FIG. 3B is a schematic diagram of a top view of a negative test result using the exemplary cassette of FIG. 1A.

FIG. 3C is a schematic diagram of a top view of two invalid test results using the exemplary cassette of FIG. 1A.

FIG. 4A is a schematic top view of a vertical swab holder before placement on a cassette.

FIG. 4B is a schematic elevation view of the vertical swab holder of FIG. 4A.

FIG. 4C is a schematic elevation view of an upper portion of a cassette designed to house the vertical swab holder of FIG. 4A and FIG. 4B.

### DETAILED DESCRIPTION

The present disclosure provides for a lateral flow assay cassette including a vertical swab holder. As used herein, the term “cassette” refers to the outer housing of a device for a lateral flow assay. Other components of the lateral flow assay may include a sample pad, a conjugate pad, a membrane, an absorbent pad. The cassette includes a sample port, which permits access to the sample pad, and a swab holder, which secures a sample swab. The cassette may include other features, such as one or more viewing ports for viewing a test and/or control line.

The present disclosure also provides an assay for an analyte conducted using the lateral flow assay cassette. The assay may be an immunoassay. The immunoassay may include one or more antibodies that specifically bind the analyte. In some examples. The immunoassay may contain more than one type of antibody that both specifically bind the antigen, or that bind separate antigens. For example, the immunoassay may contain two separate antibodies that both bind different epitopes of one antigen.

“Antibodies” is meant in a broad sense and includes immunoglobulin molecules belonging to any class, IgA, IgD, IgE, IgG and IgM, or sub-class IgA1, IgA2, IgG1, IgG2, IgG3 and IgG4 and including either kappa and lambda light chain. Antibodies include monoclonal antibodies, full length antibodies, antigen binding fragments, bispecific multispecific antibodies, dimeric, tetrameric or multimeric antibodies, single chain antibodies, domain antibodies and any other modified configuration of the immunoglobulin molecule that comprises an antigen binding fragment of the required specificity. “Antibodies” include antibodies generated using various technologies, including antibodies generated from immunized animals such as mice, rats, alpacas, rabbits or chickens, or identified from phage or mammalian display libraries.

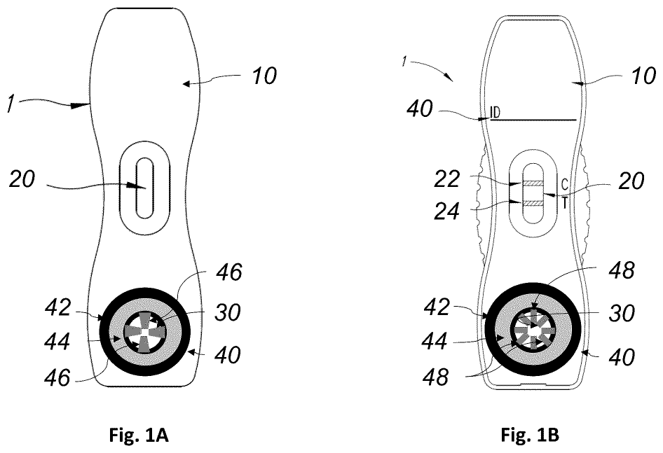

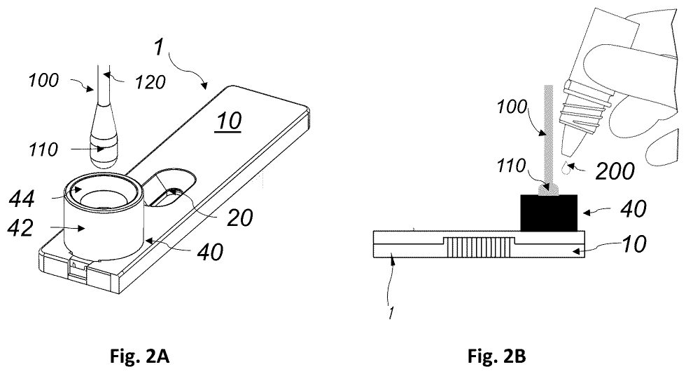

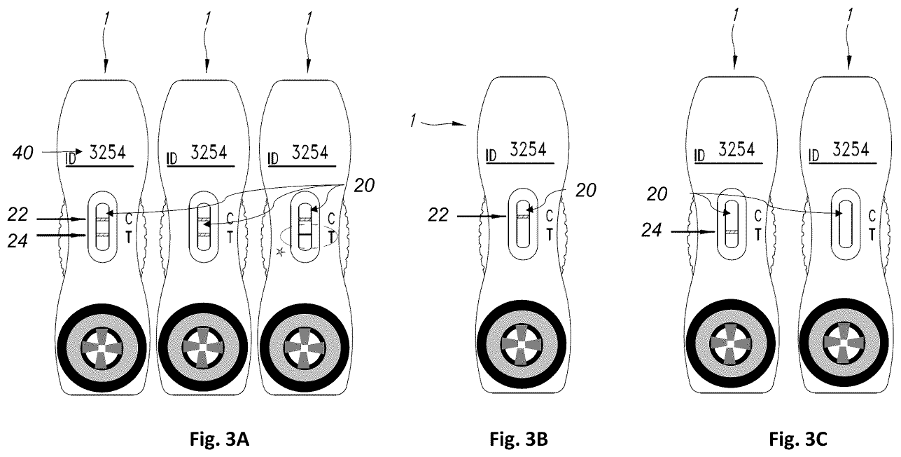

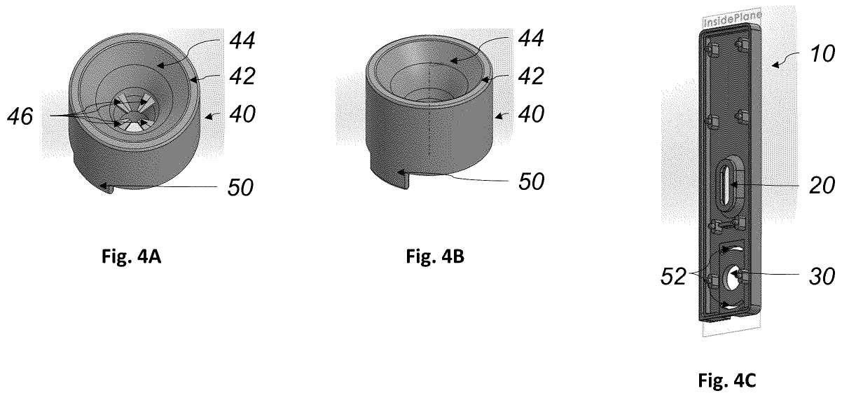

Referring now to FIG. 1A, FIG. 1B, FIG. 2A, FIG. 2B, FIG. 3A, FIG. 3B, FIG. 3C, FIG. 4A, FIG. 4B, and FIG. 4C, the lateral flow assay may be of typical construction known in the art, including lateral flow assay cassette 1, which may include an outer housing 10, a sample pad, a conjugate pad, membrane and an absorbent (or ‘wicking’) pad (not shown). The membrane may be visible through a result display window 20. The outer housing 10 may be typically made of a plastic suitable for use in lateral flow immunoassays (e.g., HIPS, ABS, etc.). The disclosed designs, in contrast, specifically hold the swab head 110 of the swab 100 either directly in contact with the rapid test sample pad or directly above the sample pad. The cassette 1 may further include a sample port 30 located above the sample pad to allow the swab head 110 to be placed directly in contract with the sample pad or directly above the sample pad.

The cassette 1 may further include a result display window 20, which allows one or more result indicators to be viewed. In some embodiments, the result indicators may be include lines. In the exemplary embodiments of FIG. 1B, FIG. 3A, FIG. 3B, and Fig. C, the result display window 20 may display a control line 22, which indicates that the assay is working as expected, as well as a test line 24, which indicates a result, such as a positive or negative result. In some embodiments, the control line 22, may correspond to a portion of the membrane that reacts with a control component of the assay, such as an antibody bound to an antigen, to form a visible indicator.

The cassette 1 may include other features to facilitate its use, such as a unique identifier. The tracking identifier may be a bar code or other computer readable identifier, or a simple location to write a tracking number or paste a label with the tracking number, such as ID line 40.

The cassette 1 further includes a vertical swab holder 40, which includes an outer portion 42 and an inner portion 44. In some embodiments, such as those illustrated in FIG. 1A, FIG. 1B, FIG. 2A, FIG. 3A, FIG. 3B, FIG. 3C, FIG. 4A, and FIG. 4B, the outer portion 42 has a height (dimension in the vertical direction relative to the cassette 1) that is greater than the height of the inner portion 44. In some embodiments, such as those illustrated in FIG. 4A and FIG. 4B, the inner portion 44 slopes downward from the outer portion 42 towards the sample port 30. This downward slope may facilitate movement of the sample buffer 200 onto the sample pad.

In the embodiment shown in FIG. 1A, FIG. 3A, FIG. 3B, FIG. 3C, FIG. 4A, and FIG. 4B, the vertical swab holder 40 includes a plurality of flexible protrusions 46 that securely hold swabs 100 of varying dimensions in a vertical position with respect to the cassette 1 by engaging with the swab head 110 of varying dimensions. The flexible protrusions 46 may be formed contiguously with or affixed to the inner portion 44 of the vertical swab holder 40. In the embodiments illustrated, the flexible protrusions 46 are flaps.

A swab 100 is in a “vertical position” with respect to an assay cassette 1 when an axis that runs from the swab head end of the swab 100, to the opposite end of the swab stem 120, is position at an angle in a range between 60 degrees and 90 degrees, 70 degrees and 90 degrees, 80 degrees and 90 degrees, 85 degrees and 90 degrees, or 88 degrees and 90 degrees with respect to a plane defined by the two largest dimensions of the outer housing 10 of the cassette 1.

In the embodiment shown in FIG. 1B, the vertical swab holder 40 includes a plurality of ribs 48 that securely hold swabs 100 of varying dimensions in a vertical position with respect to the cassette 1 by engaging with the swab head 110 of varying dimensions. The ribs 48 may be formed contiguously with or affixed to the inner portion 44 of the vertical swab holder 40. In some embodiments, the ribs 48 may be rigid to more securely hold the swab head 110, and may be sized to securely hold a swab head of a particular diameter, or range of diameters. In other embodiments, the ribs 48 may be deformable to hold swabs with a larger variety of dimensions, and, in particular, swab heads 110 with a larger range of diameters, than if ribs 48 are not deformable.

In still other embodiments (not shown) vertical swab holder 40 may include any internal features sufficient to securely hold a swab 100 in a vertical position with respect to the cassette 1 by engaging with the swab head 110. For example, in some embodiments inner portion 44 may simply include an orifice above the sample pad that has a diameter that allows the inner portion 44 to securely hold swabs 100 of varying dimensions in a vertical position with respect to the cassette 1 by engaging with the swab head 110 of varying dimensions. In other embodiments, rigid or flexible elements having different shapes may extend from inner portion 44 to engage the swab head 110.

When flexible elements, such as flexible protrusions 46 or flexible ribs 48, are used to secure the swab 100 in a vertical position with respect to the cassette 1, the flexible elements may be capable of being bent upon insertion of a swab head 110 with ordinary manual force, and a spring-like force from the flexible elements may provide sufficient friction to hold swab heads 110 of a variety of dimensions.

When rigid elements, such as rigid ribs 48, are used to secure swab 100 in a vertical positon with respect to the cassette 1, the rigid elements may slightly deform adjacent sections of the swab head 110 to allow friction and pressure to hold swab heads 110 of a variety of dimensions.

A variety of types of sample collection swabs 100 are known in the art. Any appropriate swab 100 may be used. Swabs 100 that may be suitable for use include, but are not limited to, nasal, nasopharyngeal, mid-turbinate, and saliva/sputum/oropharyngeal swab types. Swabs 100 typically include a swab head 110 attached to a swab stem 120.

The swab head 110 is formed of a material to which the desired analyte does not adsorb. In some embodiments, the swab head 110 is formed of polyester, spun polyester, or another suitable material that ensures for the thorough release of the target analyte. In some embodiments, the swab head has a diameter ranging from 1 mm to 10 mm, from 1 mm to 5 mm, from 1 mm to 3 mm, from 3 mm to 10 mm, or from 3 mm to 5 mm.

In some embodiments, the outer portion 41, the inner portion 44, elements that engage the swab head 110, such as the flexible protrusions 46 or the ribs 48, or any combinations thereof may be formed from the same material as the outer housing 10 of the cassette 1, which allows for simpler and more cost-effective manufacture. Given the rigidity of the outer housing 10, this embodiment may be most compatible with rigid elements that engage the swab head 110, such as rigid ribs 48.

The cassette 1 includes a sample port 30 that allows access to the sample pad. The vertical swab holder 40 secures the swab 100 in a position that allows the swab head 110 to rest within the sample port 40, directly over or in contact with the sample pad.

A lateral flow assay cassette 1 as provided herein may be used for detection of any analyte that may be present in a sample obtained using a swab 100. In some embodiments, the analyte is RNA, DNA, or protein. In some embodiments, analytes may be indicative of the presence of an infectious agent, including a virus, bacteria, or fungus. Exemplary infectious agents include, but are not limited to respiratory viruses such as coronavirus, including, SARS-CoV-2, influenza, and RSV, as well as gastrointestinal viruses. Other exemplary infectious agents include respiratory, gastrointestinal, and skin bacteria or fungi, such as tuberculosis, S. aureus, such as MRSA, and yeast. Specific exemplary infectious agents include, Human coronavirus, 229E; Human coronavirus, OC43; Human coronavirus, NL63; MERS-coronavirus; SARS-CoV-2; Adenovirus 21; Human Metapneumovirus (hMPV); Parainfluenza virus 1; Parainfluenza virus 2; Parainfluenza virus 3; Parainfluenza virus 4a; Influenza A; Influenza B; Enterovirus D68; Respiratory syncytial virus; Rhinovirus 40; Haemophilus influenza; Streptococcus pneumonia; Streptococcus pyogenes; Candida albicans; Bordetella pertussis; Mycoplasma pneumonia; Chlamydia pneumonia; Legionella pneumophila; and Staphylococcus aureus.

It will be appreciated that the sample type and the location from which the swab sample is obtained will vary depending on the infectious agent to be detected. Although nasal swab specimens are used in many of the exemplary embodiments herein, the specimen may be any biological tissue or material, a portion of which may be collected on a swab. The biological tissue or material may be collected directly from its source onto the swab, or it may be removed from its source and prepared or treated in any manner prior to collection on the swab. Biological secretions or emissions, such as nasal or ear secretions, sputum, saliva, vaginal secretions, pus, blood, urine, or feces may be collected on a swab. Example biological locations that may be swabbed directly include the nose, nasopharyngeal cavity, the oropharyngeal cavity, the mid-turbinate region, mouth, tongue, throat, teeth, gums, tonsils, outer ear canal, skin, wounds, lesions, urethra, vagina, cervix, anus, or rectum. For some assays, particularly those looking for environmental contamination, the sample may be taken from an inanimate object, such as an environmental surface, or it may constitute a sample deposited from the air. Specimens, such as secretions or emissions, from all of these locations may also be obtained and then placed on a swab, as may specimens obtained through more invasive procedures, such a bronchoalveolar lavage samples, or surgical procedures.

In example embodiments, the swab 100 may be sterile prior to specimen collection. The swab head 110 may be made of any of a variety of materials, provided that the material is suitable for collecting the specimen and preserving it sufficiently to be used in the intended assay. For example, the swab head 110 may be cotton. Similarly, the swab stem 120 may be made of any durable material, such as paper, cardboard, wood, or plastic.

Collection of the specimen on the swab 100 may be in any setting, including a hospital setting, a clinical setting, a field setting, or an at-home setting. The assay may be conducted within 0 minutes, 1 minute, 2 minutes, 5 minutes, 10 minutes, 30 minutes, 1 hour, 2 hours, 4 hours, 6 hours, 8 hours, 12 hours, 1 day, 3 days, 2 days, 4 days, 5 days, 1 week, or 1 month of collecting the specimen on the swab 100. The swab 100 may be stored in appropriate conditions to maintain sample integrity. For example, the swab 100 may be placed in a protective container, such as a glass, plastic, paper, or cardboard container or holder. As another example, the swab 100 may be refrigerated. The swab 100 may also be placed in or treated with any of a number of buffers, preservatives, lysis agents, and other agents or materials able to preserve sample integrity until the assay may be conducted.

Referring now to an assay performed as in FIG. 2A, FIG. 2B, FIG. 3A, FIG. 3B, and FIG. 3C, the cassette 1 may be labeled with a unique identifier, for example on ID line 40, or it may already be labeled with a unique identifier prior to use. In either case, the unique identifier may be associated with a sample source, such as a subject.

The swab head 110 is contacted with the sample source in a manner sufficient to obtain a sample for the assay.

FIG. 2A shows a method of placing a swab 110 in the cassette 1. Specifically, the swab head 110 is lowered into vertical sample port 40 such that the swab 100 is securely held in a vertical position with respect to the cassette 1.

As shown in FIG. 2B, after correct placement of the swab head 110, while the swab 100 is held securely above or directly in contact with the sample pad, a sample buffer 200, such as a lysis buffer may then be added directly to the swab 100 to release the target analyte onto the sample pad. In some embodiments, the sample buffer 200 is placed in the vertical swab holder 40. In embodiments where the vertical swab holder 40 includes a sloping inner portion 44, the slope may funnel the sample buffer 200 onto the swab head 110 and into the sample port 30.

The vertical swab holder 40 may have diameter sufficient to facilitate addition of the sample buffer 200. For example, the vertical swab holder 40 may have a diameter in a range from 1 cm to 5 cm, 1 cm to 4 cm, 1 cm to 3 cm, 1 cm to 2 cm, 2 cm to 5 cm, 2 cm to 4 cm, 2 cm to 3 cm, 3 cm to 5 cm, 3 cm to 4 cm, or 4 cm to 5 cm, or a diameter of at least 1 cm, at least 2 cm, at least 3 cm, at least 4 cm, or at least 5 cm.

In an exemplary embodiment, the sample buffer 200 may be dispensed from a dropper bottle as shown in FIG. 2B by placing the tip of dropper bottle above the vertical swab holder 40 and dropping the sample buffer on into the swab holder 40, onto the swab head 110, or both. In some embodiments, a set number of drops of the sample buffer may be dispensed

In some embodiments, sample buffer alone will be sufficient to release and run the entire lateral flow immunoassay. In other embodiments, the sample buffer may be followed by a chase buffer. A typical lysis buffer type of sample buffer may include 1% Triton X-100 or 1-2% Igepal. Chase buffer may include casein or other proteinaceous blocking reagents and/or non-ionic detergents. After the sample buffer is added directly to the swab head 110, both excess buffer and capillary action will be sufficient to transfer the (now processed) analyte from the swab head 110 onto the sample pad and the rapid test process will continue as normal. If chase buffer is desired or required for the assay, the chase buffer may be added to directly to the vertical sample port 40, the swab head 110, or both after the sample buffer 200, for example in a manner similar to that shown in FIG. 2B, but with a chase buffer dropper bottle in place of the sample buffer dropper bottle.

After a period of time sufficient for they assay to be completed, but not so long as to allow results to become inaccurate, results of the assay are viewed in the result display window 20. In the example of FIG. 3A, FIG. 3B, and FIG. 3C, the results include a control line 22, which is visible if the assay functions as expected. As shown in FIG. 3A and FIG. 3B, the control line 22 is visible if the assay functioned as expected. As shown in FIG. 3C, the control line 22 is not visible if the assay did not function as expected, regardless of whether the test line 24 is visible. Only assay results in which control line 22 are visible may be considered a positive or negative result. Results in which the control line 22 is not visible are invalid.

As shown in FIG. 3A and FIG. 3B, the results also include a test line 24, which is visible or not visible depending on whether the analyte is present. In the example of FIG. 3A, the test line 24 is visible if the analyte is present. The intensity of the test line may vary depending on the amount of analyte (as illustrated in FIG. 3A, which slows test lines of varying intensities), but any visible test line 24 is indicative of a positive result. In the example of FIG. 3B, the test line 24 is not visible if the analyte is not present, which is indicative of a negative result. Further, in the example of FIG. 3C, the control line 22 is not visible if the assay did not function as expected and regardless of whether test line 24 is visible, such results are invalid.

Results may be read visually by any human with sufficient visual acuity to resolve lines 22 and 24. Results may also be read digitally using a digital camera or other light detector in communication with a processor programmed to recognize and interpret lines 22 and 24 using digital information from the camera. For example, the results may be read digitally using a smartphone, a camera connected to a laboratory computer, or a dedicated reader.

The accuracy of the test depends upon the underlying lateral flow immunoassay performance and the concentration of the analyte that is removed from the swab 100. In a preferred embodiment, assay results may be understood and processed with a clinician. In another embodiment, assay results may be understood and processed by any person or machine.

Working cassettes have been developed with 3D printers—permitting iterations of the design to ensure the plastic housing works as desired. In a preferred embodiment, the cassette is engineered using 3D CAD design and 3D printers, and takes into account the principles of lateral flow immunoassay and sample type processing steps.

The outer housing 10 and the vertical swab holder 40 may be formed to facilitate manufacture of the cassette 1. In some embodiments, the outer housing 10 and the vertical swab holder 40 may be formed contiguously. In other embodiments, such as those illustrated in FIG. 4A, FIG. 4B, and FIG. 4C, the outer housing 10 and the vertical swab holder 40 are formed separately, then associated with one another during manufacture of the cassette 1. For example, the outer housing 10 and the vertical swab holder 40 may be associated with one another via a post and groove joint. In the embodiment shown in FIG. 4A, FIG. 4B, and FIG. 4C, the vertical swab holder 40 includes at least one post 50, which fits into at least one groove 52 in the outer housing 10 to secure the vertical swab holder 40 to the outer housing 10.

The terms “specimen” and “sample” are used interchangeably in this specification. Similarly, the terms “test” and “assay” are used interchangeably.

Elements of the different embodiments disclosed herein, including those in the Examples, may be combined with one another, Furthermore, not all aspects described in connection with a given embodiment are required for that embodiment to function and many aspects are provided merely as details to assist in implementing the disclosure.

The various embodiments described above can be combined to provide further embodiments. Aspects of the embodiments can be modified, if necessary to employ concepts of the various patents, applications and publications to provide yet further embodiments.

All numerical values provided herein, with the exception of numerical values reflecting actual data, include values “about” the recited value, such as or within +/−5% of the recited value.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A lateral flow assay cassette comprising:
a sample pad;
a sample port that allows access to the sample pad; and
a vertical swab holder that secures a swab having a stem and a head in a vertical position with respect to the cassette by engaging the head of the swab.

2. The lateral flow assay cassette of claim 1, wherein the vertical swab holder includes an outer portion and an inner portion which contains the sample port in an orifice of the inner portion.

3. The lateral flow assay cassette of claim 2, wherein the outer portion has a height that is greater than a height of the inner portion.

4. The lateral flow assay cassette of claim 1, wherein the vertical swab holder comprises a plurality of flexible elements that secure the swab in a vertical position.

5. The lateral flow assay cassette of claim 4, wherein the plurality of flexible elements comprise a plurality of flexible protrusions that protrude from the inner portion.

6. The lateral flow assay cassette of claim 4, wherein the plurality of flexible elements comprise a plurality of flexible ribs that protrude from the inner portion.

7. The lateral flow assay cassette of claim 1, wherein the vertical swab holder comprises a plurality of rigid elements that secure the swab in a vertical position.

8. The lateral flow assay cassette of claim 7, wherein the plurality of rigid elements protrude from the inner portion.

9. The lateral flow assay cassette of any one of claim 4, wherein the plurality of flexible elements or the plurality of rigid elements are sized to engage swab heads of varying dimensions.

10. A method of detecting an analyte, the method comprising:
a) providing a lateral flow assay cassette having a sample pad, a sample port that allows access to the sample pad, and a vertical swab holder that secures a swab having a stem and a head in a vertical position with respect to the cassette by engaging the head of the swab;
b) collecting a sample from a subject using the swab;
c) placing the swab stem into the swab holder in a vertical position with respect to the cassette such that the swab head is in the sample port directly above the sample pad or directly in contact with the sample pad; and
d) adding an appropriate amount of sample buffer to the swab head or vertical swab holder, or both.

11. The method of claim 10, wherein the sample is from a nose, nasopharyngeal cavity, the oropharyngeal cavity, the mid-turbinate region, mouth, tongue, throat, teeth, gums, tonsils, outer ear canal, skin, wounds, lesions, urethra, vagina, cervix, anus, or rectum sample.

12. The method of claim 10, wherein the sample is a nasal or ear secretion, sputum, saliva, a vaginal secretion, pus, blood, urine, feces, a bronchoalveolar lavage sample, or a surgical sample.

13. The method of claim 12, wherein the detection of the analyte indicates the presence of an infectious agent in the subject.

14. The method of claim 13, wherein the infectious agent is Human coronavirus, 229E; Human coronavirus, OC43; Human coronavirus, NL63; MERS-coronavirus; SARS-CoV-2; Adenovirus 21; Human Metapneumovirus (hMPV); Parainfluenza virus 1; Parainfluenza virus 2; Parainfluenza virus 3; Parainfluenza virus 4a; Influenza A; Influenza B; Enterovirus D68; Respiratory syncytial virus; Rhinovirus 40; Haemophilus influenza; Streptococcus pneumonia; Streptococcus pyogenes; Candida albicans; Bordetella pertussis; Mycoplasma pneumonia; Chlamydia pneumonia; Legionella pneumophila; or Staphylococcus aureus.

15. The method of claim 10, wherein the analyte is RNA, DNA, or protein.

16. The method of claim 10, further comprising detecting the analyte within 30 minutes of adding the sample buffer to the swab head or vertical swab holder, or both.

17. The method of acclaim 10, further comprising:
e) introducing sample from the swab head to the sample pad;
f) running the assay; and
g) producing an assay result.

18. The method of claim 10, further comprising adding an appropriate amount of chase buffer to the swab head after step (d).

19. A kit comprising:
a lateral flow assay cassette comprising:
a sample pad;
a sample port that allows access to the sample pad; and
a vertical swab holder that secures a swab having a stem and a head in a vertical position with respect to the cassette by engaging the head of the swab;
a swab having a stem with a diameter sized to be secured by the swab holder; and
an instruction sheet.

20. The kit of claim 19, further comprising sample buffer.
